(19) United States
(12) Patent Application Publication
(10) Pub. No.: US 2006/0078518 A1
(43) Pub. Date: Apr. 13, 2006

(54) Cosmetic compositions containing fluorescent colorants for natural skin appearance

(76) Inventors: Stewart T. Elder, Butler, NJ (US); Colin J. H. Morton, Basel (CH); Christina L. Andrianov, Monroe, NY (US)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 11/205,570

(22) Filed: Aug. 17, 2005

Related U.S. Application Data
(60) Provisional application No. 60/603,591, filed on Aug. 23, 2004.

(51) Int. Cl. A61K 8/49; C07D 491/02

(52) U.S. Cl. 424/63; 546/47; 424/64

(57) ABSTRACT

Cosmetic or personal care compositions which comprise at least one fluorescent colorant other than white and at least one non-fluorescent colorant, are useful in preparing colored cosmetic or personal care preparations providing an enhanced skin appearance.

The present invention relates to the use of fluorescent colorants in cosmetic compositions. More particularly this invention utilizes a system comprising at least one fluorescent colorant other than white in combination with at least one standard (non-fluorescent) colorant that provides unusual fluorescent effects. The final cosmetic product remains fluorescent. Cosmetic applications include all facial makeup and skin products where a natural skin appearance is desired, as well as eye products and any other personal care products that use colorants such as lipsticks.

The term fluorescent colorant embraces all fluorescent organic coloring matter and includes both pigments and dyes.

It is well known to add an optical brightener (fluorescent whitening agent) to cosmetic compositions. One recent example of this is U.S. Patent Application Publication No. 2002/0192260 A1, which discloses optically-activated particles for use in cosmetic preparations to reduce the visual perception of skin imperfections. The optically-activated particles are of various substrates such as thermoplastic polymers, natural materials, regenerated cellulose, metals and minerals which have an optical brightener chemically bonded to the substrate particles to form integral units in the form of optically-activated particles. Said particles are taught to be useful for diffusing and emitting light to reduce the visual perception of cellulite, shadows, skin discolorations and wrinkles.

However, optical brighteners only add or contribute to the blue region of the visible spectrum, which at best would give a whiter appearance to the skin.

However, it has now been found that utilizing fluorescent colors other than white, in particular those having a yellow and/or reddish hue, adds color to regions of the skin's natural color that are prominent in the skin's visible spectrum. The result is unique color effects that are unachievable by other methods.

Thus, the colorant of the invention comprises at least one fluorescent colorant other than white in addition to at least one standard (non-fluorescent) colorant. The final composition still retains fluorescent activity.

A general introduction and overview of organic colorants is provided in, W. Herbst, K. Hunger; Industrial Organic Pigments, 2^{nd} Ed, VCH Verlagsgesellschaft, Weinheim, 1997, and a brief overview of fluorescent colorants in particular is given in B. M. Krasovitskii, B. M. Bolotin; Organic Luminescent Materials, 1988, VCH Weinheim.

A variety of colorants, from many chemical classes, show solid and/or solution state fluorescence. In principle, absorption of light energy excites molecules in these colored materials to their excited states. When deactivation of these excited states takes place via radiative decay, resulting in emission of light at a longer wavelength to the absorbed light, then fluorescence is said to have occurred. To the observer, this effect provides an unusual brilliance to the colorant, beyond that of traditional colorants employed in cosmetics. This brilliance is caused by both the ultraviolet and short-wavelength components of visible light.

Typical fluorescent colorants (both pigments and dyes), other than traditional fluorescent whitening agents, can be used in the present invention. They include for example, but are not limited to, materials of the following chemical classes:
a) perylene derivatives of the general formulae 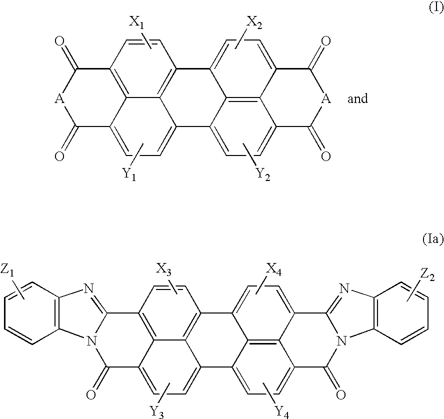
   or its regioisomer,
   wherein A=O, N—H, N-alkyl or N-aryl and X₁, X₂, X₃, X₄, Y₁, Y₂, Y₃, Y₄, Z₁, Z₂ are independently halogen, O-alkyl or O-aryl, for example Pigment Red 224;
b) fluoresceins of the general formula 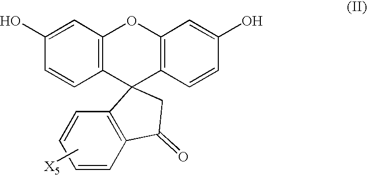
   wherein X₅═Cl, Br or I, for example Solvent Yellow 94, CI 45350;
c) rhodamines of the general formula 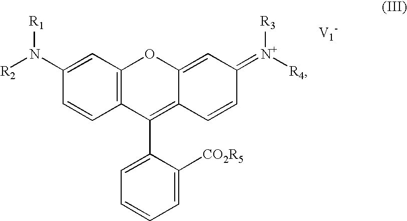
   wherein the R₁, R₂, R₃, R₄, R₅ groups are independently H or C₁-C₁₂alkyl, and V₁═Cl, Br or I, for example Basic Red 1, CI 45160;
d) pyronines of the general formula 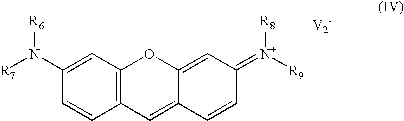
wherein R₆, R₇, R₈ and R₉ groups are independently H or C₁-C₁₂alkyl, and V₂═Cl, Br or I, for example Pyronin G, CI 45005;
e) naphthalic anhydride derivatives of the general formula 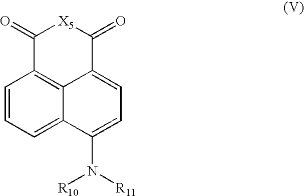
   wherein X₅═O, N—H, N-alkyl or N-aryl and R₁₀ and R₁, are independently H, alkyl or aryl, for example Solvent Yellow 43;
f) benzoxanthenes and benzthioxanthenes of the general formulae 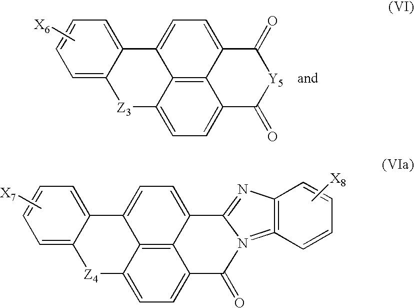
   or its regioisomer,
   wherein X₆, X₇ and X₈ are independently O-alkyl, O-aryl or halogen, Y₅═O, N—H, N-alkyl or N-aryl and Z₃ and Z are independently O or S, for example Solvent Yellow 98;
g) benzanthrone derivatives of the general formula 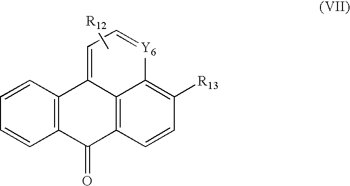
   wherein Y₆═C or N and R₁₂ and R₁₃ are independently halogen, alkoxy, amino or alkylamino and when Y₆═C, Y₆ plus R₁₃ can form a 6-membered ring, optionally fused to an arylene group, for example Solvent Orange 63 of the formula (VIIa); 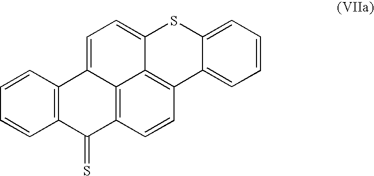
h) coumarins of the general formula 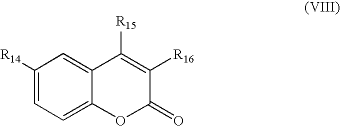
   wherein R₁₄—H, alkoxy, amino or alkylamino, and R₁₅ and R₁₆ are independently H, alkyl or aryl; for example solvent yellow 160;
i) isoindolo-[2,1-a]-benzimidazolone derivatives of the general formula 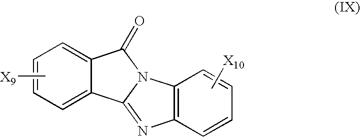
   wherein X₉ and X₁₀ and are independently hydrogen or halogen, for example 1,2,3,4,7-pentachloro-11H-isoindolo-[2,1-a]-benzimidazol-11-one of the formula 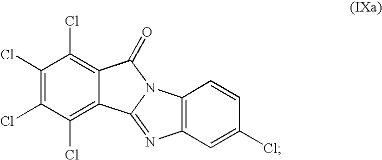
j) diphenyl maleimides of the general formula 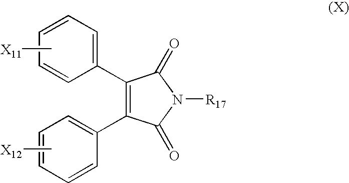
   wherein R₁₇=optionally substituted alkyl or aryl and X₁₁, and X₁₂ are independently H, alkyl, aryl, O-alkyl, O-aryl or halogen;
k) azo pigments of the general formula 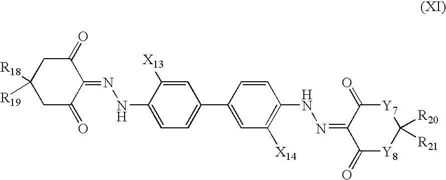
   wherein X₁₃ and X₁₄ are independently halogen or alkoxy, Y₇ and Y₈ are independently CH₂ or 0 and R₁₈, R₁₉, R₂₀ and R₂, are independently optionally substituted alkyl or aryl;
l) diketopyrrolo[3,4-c]pyrroles (abbreviated to DPP) of the general formula 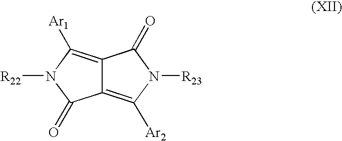
   wherein Ar₁ and Ar₂ are aryl radicals and R₂₂ and R₂₃ are independently optionally substituted alkyl or aryl radicals;
m) condensation products of aromatic or heteroaromatic aldehydes with barbituric acid derivatives,
   and mixtures thereof.

Preferred classes of fluorescent colorants for the present invention are those having a yellow and/or reddish hue. They include condensation products of N,N′-di(C₁-C₈alkyl)amino-benzaldehyde and barbituric acid such as those disclosed in Chem. Berichte, 39, 2166, (1906), perylene derivatives of the formula (I) and (Ia) such as those disclosed in DE 42 25 031, diphenyl maleimides of the formula (X) such as those disclosed in U.S. Pat. No. 6,508,957, benzoxanthenes and benzthioxanthenes of the formulae (VI) and (VIa) such as those disclosed in EP 1 172 418 A2 and corresponding U.S. Pat. Nos. 6,462,128 and 6,559,306 and similar compounds, N,N′-disubstituted DPPs of the formula (XII) and the isoindolo-[2,1-a]-benzimidazolone of the formula 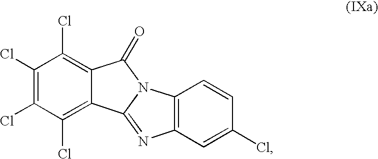
and mixtures thereof, wherein the substituents in the formulae are as defined above.

N,N′-disubstituted DPPs are disclosed or at least generically mentioned in WO 2003/022848, U.S. Pat. No. 6,603,020, WO 2000/033795, U.S. Pat. No. 6,372,202, U.S. Pat. No. 6,042,842, WO 2004/033563 and U.S. Pat. Nos. 6,113,683 and 6,160,037.

WO 2003/022848 (Ciba), relates to a new process for the preparation of certain diketopyrrolopyrroles which in particular makes it possible to obtain DPPs which are unsymmetrically substituted at the nitrogen atoms of the DPP basic unit in an acceptable yield, and to the novel unsymmetrically substituted DPPs produced thereby. Cosmetic use is suggested but not shown, and no combination with a non-fluorescent colorant for cosmetic use is described.

U.S. Pat. No. 6,603,020 (Ciba), relates to certain fluorescent DPPs of the formula I′ 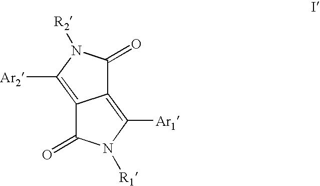
wherein R₁′ and R₂′, independently from each other, stand for C₁-C₂₅-alkyl, preferably C₁-C₂₅-alkyl, which is substituted with a functional group capable of increasing the solubility in water such as a tertiary amino group, —SO₃⁻, or PO₄², allyl which can be substituted one to three times with C₁-C₃alkyl or optionally substituted phenyl or 1- or 2-naphthyl, and Ar₁′ and Ar₂′, independently from each other, stand for an aryl or heteroaryl group. Cosmetic use is suggested but not shown, and no combination with a non-fluorescent colorant for cosmetic use is described.

WO 2000/033795 (Ciba), relates to cosmetic preparations or formulations for making-up the lips or the skin and for coloring the hair or the nails, comprising certain red DPP pigments of formulae 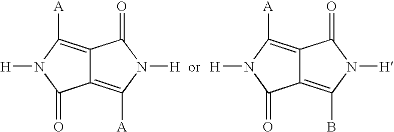
wherein A and B are each independently of the other an aryl or heteroaryl group. Since the N-atoms are unsubstituted, the pigments have, for practical purposes, no fluorescence, i.e. they are used merely as conventional non-fluorescent red colorants.

U.S. Pat. No. 6,372,202 (L'Oreal), relates to cosmetic compositions of certain special DPPs of the formula

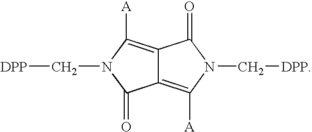

There is no teaching these DPPs are fluorescent, and no combination with a non-fluorescent colorant is shown.

U.S. Pat. No. 6,042,842 (L'Oreal) relates to cosmetic compositions of a red DPP pigment with an intense and saturated color, which does not generate free radicals. While the DPPs are broadly disclosed, only Pigment Red 254 (CI56110) is used. Since the N-atoms are unsubstituted in Pigment Red 254, the pigment has, for practical purposes, no fluorescence, i.e. it is used merely as a conventional non-fluorescent red colorant. Additionally, no combination with a non-fluorescent colorant is shown.

WO 2004/033563 (Ciba) relates to a method of producing colored carrier particles wherein, inter alia, certain intermediate DPPs having labile groups on the N atoms are deposited on particles, e.g. metal flakes or layered silicates, and then converted to DPPs in which the N-atoms are unsubstituted. Cosmetic use of the produced colored carrier particles, but not the fluorescent intermediates, is suggested but not shown.

U.S. Pat. No. 6,113,683 (Ciba) discloses colored pearlescent pigments comprising a pearlescent pigment and a second pigment, and the use of an adhesion promoter for the mutual adsorbance of the pearlescent pigment and the second pigment. Since the N-atoms in the DPP pigments employed are unsubstituted, the DPP pigments have, for practical purposes, no fluorescence. Cosmetic use of the colored pearlescent pigments is suggested but not shown.

U.S. Pat. No. 6,160,037 (Ciba) relates to mixtures containing certain pigment intermediates, inter alia DPPs, having labile groups on the N-atoms and polymerizable substances, and to a method of producing colored polymeric materials, wherein the fluorescent DPP pigment intermediates are converted via reactive extrusion into DPPs in which the N-atoms are unsubstituted. Cosmetic use of the pigment compositions is eluded to, but not shown, and no combination with a non-fluorescent colorant for cosmetic use is described.

U.S. Pat. No. 6,203,909 (Ciba) relates to a composite pigment which consists of a colorant and a substrate, using a substrate (S) and a colorant (C), which are each coated with ions or ionizable compounds having a charge rotating in the same direction, and, if desired, of additional ions or ionizable compounds having a change rotating in the same direction as a layer material (L), wherein either (I) the sign of the change of the coating of S, or the sign of the charge of the coating of S and of the charge of L, which is the same, is opposite to that of charge of the coating of C, or (II) the sign of charges of the coatings of S and C is the same and is opposite to that of the charge of L. The colorant may be a DPP pigment. Cosmetic use is suggested but not shown, and no combination with a non-fluorescent colorant for cosmetic use is described.

Particularly preferred fluorescent colorants for the present invention are condensation products of p-N,N′-di(C₁-C₃alkyl)aminobenzaldehyde and barbituric acid, for example the condensation products of p-N,N′-di(methyl)aminobenzaldehyde and barbituric acid. These latter products are known and have the CAS numbers 1753-475 and 152734-34-4. They are used alone or preferably in combination.

Also particularly preferred are fluorescent colorants from the benzoxanthene class, especially the compound of the formula

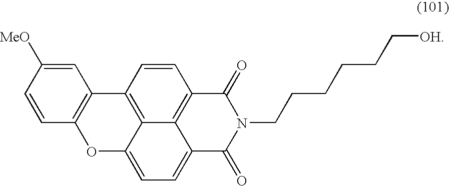

Also particularly preferred are mixtures comprising condensation products of p-N,N′-di(C₁-C₃alkyl)aminobenzaldehyde and barbituric acid and fluorescent colorants from the benzoxanthene class, especially the condensation products of p-N,N′-di(methyl)aminobenzaldehyde and barbituric acid and the benzoxanthene of the formula (101).

As indicated previously, the colorant of the invention comprises at least one fluorescent colorant other than white in addition to at least one standard (non-fluorescent) colorant, such that the final composition still retains fluorescent activity. The inventive colorant may additionally comprise further active ingredients, for instance UV absorbers, UV reflectors, and other cosmetically acceptable ingredients and mixture thereof.

The non-fluorescent colorant may be any colorant, for instance a dye, pigment or lake, or a mixture thereof. Typical suitable non-fluorescent colorants include any organic or inorganic pigment or colorant approved for use in cosmetics by CTFA and the FDA such as lakes, titanium dioxide (in low amounts) or other conventional pigments used in cosmetic formulations.

Examples of pigments include inorganic pigments such as carbon black, D&C Red 7, calcium lake, D&C Red 30, talc lake, D&C Red 6, barium lake, talc, kaolin, mica, mica titanium, magnesium silicate; and organic pigments such as Red No. 202, Red No. 204, Red No. 205, Red No. 206, Red No. 219, Red No. 228, Red No. 404, Yellow No. 205, Yellow No. 401, Orange No. 401 and Blue No. 404. Examples of vat dyes are Red No. 226, Blue No. 204 and Blue No. 201. Examples of lake dyes include various acid dyes which are laked with aluminum, calcium or barium.

In one embodiment the colorant is an aqueous solution of a water-soluble dye. Such dyes may include FD&C Blue No. 11, FD&C Blue No. 12, FD&C Green No. 13, FD&C Red No. 13, FD&C Red No. 140, FD&C Yellow No. 15, FD&C Yellow No. 16, D&C Blue No. 14, D&C Blue No. 19; D&C Green No. 15, D&C Green No. 16, D&C Green No. 18, D&C Orange No. 14, D&C Orange No. 15, D&C Orange No. 110, D&C Orange No. 111, D&C Orange No. 117, FD&C Red No. 14, D&C Red No. 16, D&C Red No. 17, D&C Red No. 18, D&C Red No. 19, D&C Red No. 117, D&C Red No. 119, D&C Red No. 121, D&C Red No. 122, D&C Red No. 127, D&C Red No. 128, D&C Red No. 130, D&C Red No. 131, D&C Red No. 134, D&C Red No. 139, FD&C Red No. 140, D&C Violet No. 12, D&C Yellow No. 17, Ext. D&C Yellow No. 17, D&C Yellow No. 18, D&C Yellow No. 111, D&C Brown No. 11, Ext. D&C Violet No. 12, D&C Blue No. 16 and D&C Yellow No. 110.

The above dyes are well known, commercially available materials, with their chemical structure being described, e.g., in 21 C. F. R. Part 74 (as revised Apr. 1, 1988) and in the CTFA Cosmetic Ingredient Handbook, (1988), published by the Cosmetics, Toiletry and Fragrances Association, Inc. These publications are incorporated herein by reference.

The certified dyes can be water-soluble or, preferably, lakes thereof. Lakes are organic pigments prepared by precipitating a soluble dye on a reactive or absorbent stratum, which is an essential part of the pigment's composition. Most lakes are aluminum, barium or calcium derived. These insoluble pigments are used mostly in makeup products, either as powders or liquids, when a temporary color is desired that won't stain the skin (as oil-soluble dyes tend to do). The lakes are used in these products along with inorganic colors.

The following tables list currently available dyes and colorants approved for use in food, drugs and/or cosmetics. The selected colorant for use herein is preferably selected from the following exemplary lists.

TABLE I
Dyes certified for use in foods, drugs, cosmetics (FDC colors)

| | | |
|---|---|---|
| FD&C Blue No. 1 | FD&C Green No. 3 | FD&C Red No. 4 |
| FD&C Red No. 40 | FD&C Yellow No. 5 | FD&C Yellow No. 6 |

TABLE 2
Dyes certified for topically applied drugs and cosmetics

| Ext. DC Violet #2 | Ext. D&C Yellow No. 7 | Ext. D&C Violet No. 2 |
|---|---|---|
| D&C Brown No. 1 | FD&C Red No. 4 | D&C Red No. 17 |
| D&C Red No. 31 | D&C Red No. 34 | D&C Red No. 39 |
| D&C Violet No. 2 | D&C Blue No. 4 | D&C Green No. 6 |
| D&C Green No. 8 | D&C Yellow No. 7 | D&C Yellow No. 8 |
| D&C Yellow No. | D&C Orange No. 4 | D&C Orange No. 10 |
| 11 | | |
| D&C Orange No. | | |
| 11 | | |

TABLE 3
Dyes certified for drugs and foods only

| | | |
|---|---|---|
| D&C Blue No. 4 | D&C Brown No. 1 | D&C Green No. 5 |
| D&C Green No. 6 | D&C Green No. 8 | D&C Orange No. 4 |
| D&C Orange No. 5 | D&C Orange No. 10 | D&C Orange No. 11 |
| D&C Red No. 6 | D&C Red No. 7 | D&C Red No. 17 |
| D&C Red No. 21 | D&C Red No. 22 | D&C Red No. 27 |
| D&C Red No. 28 | D&C Red No. 30 | D&C Red No. 31 |
| D&C Red No. 33 | D&C Red No. 34 | D&C Red No. 36 |
| D&C Violet No. 2 | D&C Yellow No. 7 | D&C Yellow No. 8 |
| D&C Yellow No. 10 | D&C Yellow No. 11 | |

Some color additives are exempt from certification and permanently listed for cosmetic use, including aluminum powder, annatto, bronze powder, caramel, carmine, beta-carotene, dihydroxyacetone, disodium EDTA-copper, guanine (pearl essence), guaiazulene (azulene), mica, pyrophyllite, silver (for coloring fingernail polish), and the ultramarines (blue, green, pink, red & violet).

The cosmetic or personal care preparations and formulations according to the invention comprise from 0.005 to 20% by weight, for example 0.01 to 15%, and especially 0.10 to 10%, based on the total weight of the preparation of a mixture of at least one fluorescent colorant other than white and at least one non-fluorescent colorant. The fluorescent colorant other than white generally comprises from 0.005 to 2% by weight, for example 0.01 to 1.5%, and especially 0.5 to 1%, based on the total weight of the preparation, and the non-fluorescent colorant generally comprises from 0.005 to 20% by weight, for example 0.01 to 15%, and especially 0.10 to 10%, based on the total weight of the preparation. The non-fluorescent colorants listed on Tables 1-3 should be selected from those that will not quench the fluorescence in the cosmetic preparation. Suitable carriers for the cosmetic or personal care preparations and formulations according to the invention are the conventional cosmetically acceptable materials used in such compositions.

The cosmetic preparations and formulations according to the invention may be in the form of, for example, sticks, ointments, creams, emulsions, suspensions, dispersions, powders or solutions. They are, for example, lipsticks, mascara preparations, make-up for the cheeks, eyeshadows, foundations, eyeliners, facial and body powders or nail polishes.

If the preparations are in the form of sticks, for example lipsticks, eyeshadows, make-up for the cheeks or foundations, such preparations consist for a considerable part of fatty components, which may consist of one or more waxes, for example ozocerite, lanolin, lanolin alcohol, hydrogenated lanolin, acetylated lanolin, lanolin wax, beeswax, candelilla wax, microcrystalline wax, carnauba wax, cetyl alcohol, stearyl alcohol, cocoa butter, lanolin fatty acids, petrolatum, petroleum jelly, mono-, di- or tri-glycerides or -fatty esters that are solid at 25° C., silicone waxes, such as methyloctadecane-oxypolysiloxane and poly(dimethylsiloxy)stearoxysiloxane, stearic acid monoethanolamine, colophane and derivatives thereof, such as glycol abietates and glycerol abietates, hydrogenated oils that are solid at 25° C., sugar glycerides and oleates, myristates, lanolates, stearates and dihydroxy stearates of calcium, magnesium, zirconium and aluminium.

The fatty component may also consist of a mixture of at least one wax and at least one oil, in which case the following oils, for example, come into consideration: paraffin oil, purcellin oil, perhydrosqualene, sweet almond oil, avocado oil, calophyllum oil, castor oil, sesame oil, jojoba oil, mineral oils having a boiling point of approximately from 310 to 410° C., silicone oils, such as dimethylpolysiloxane, linoleic alcohol, linolenic alcohol, oleyl alcohol, cereal grain oils, such as wheatgerm oil, isopropyl lanolate, isopropyl palmitate, isopropyl myristate, butyl myristate, cetyl myristate, hexadecyl stearate, butyl stearate, decyl oleate, acetyl glycerides, octanoates and decanoates of alcohols and polyalcohols, for example of glycol and glycerol, ricinoleates of alcohols and polyalcohols, for example of cetyl alcohol, isostearyl alcohol, isocetyl lanolate, isopropyl adipate, hexyl laurate and octyidodecanol.

The fatty components in such preparations in the form of sticks may generally account for up to 99.91% by weight of the total weight of the preparation.

The cosmetic preparations and formulations according to the invention may additionally comprise further constituents, for example glycols, polyethylene glycols, polypropylene glycols, monoalkanolamides, undyed polymeric, inorganic or organic fillers, preservatives, UV filters and reflectors or other adjuvants and additives conventionally employed in cosmetics.

Such further constituents are, for example, a natural or a synthetic or a semi-synthetic di- or tri-glyceride, a mineral oil, a silicone oil, a wax, a fatty alcohol, a Guerbet alcohol or an ester thereof, a lipophilic functional cosmetic active ingredient including sunscreens, or a mixture of such substances.

Further constituents include lipophilic functional cosmeticallly active ingredients suitable for skin cosmetics, an active ingredient combination or an active ingredient extract that is an ingredient or a mixture of ingredients approved for dermal or topical application. The following may be mentioned by way of example:
active ingredients that offer protection against sunlight (UV filters): suitable active ingredients are filter substances (sunscreens) that are able to absorb UV radiation from sunlight and convert it into heat. According to the desired action, the following light-protection agents are preferred: light-protection agents that selectively absorb sunburn-causing high-energy UV radiation in the range of approximately from 280 to 315 nm (UV-B absorbers) and transmit the longer-wave range of approximately from 315 to 400 nm (UV-A range), as well as light-protection agents that absorb only the longer-wave radiation of the UV-A range of from 315 to 400 nm (UV-A absorbers).
   Suitable light-protection agents are, for example, organic UV absorbers from the class of the p-aminobenzoic acid derivatives, salicylic acid derivatives, benzophenone derivatives, dibenzoylmethane derivatives, diphenyl acrylate derivatives, benzofuran derivatives, polymeric UV absorbers comprising one or more organosilicon radicals, cinnamic acid derivatives, camphor derivatives, trianilino-s-triazine derivatives, phenylbenzimidazole-sulfonic acid and salts thereof, menthyl anthranilates, benzotriazole derivatives, and/or an inorganic micropigment selected from aluminum oxide- or silicon dioxide-coated TiO₂, zinc oxide or mica;
active ingredients providing protection against chemical and mechanical influences: these include all substances that form a barrier between the skin and external harmful substances, for example paraffin oils, silicone oils, vegetable oils, PCL products and lanolin for protection against aqueous solutions, film-forming agents, such as sodium alginate, triethanolamine alginate, polyacrylates, polyvinyl alcohol or cellulose ethers against the effect of organic solvents, or substances based on mineral oils, vegetable oils or silicone oils as “lubricants” against severe mechanical stresses on the skin;
moisturizing substances: the following substances, for example, are used as moisture-controlling agents (moisturizers): sodium lactate, urea, alcohols, sorbitol, glycerol, propylene glycol, collagen, elastin or hyaluronic acid;
active ingredients having a keratoplastic effect: benzoyl peroxide, retinoic acid, colloidal sulfur and resorcinol;
antimicrobial agents, for example triclosan or quaternary ammonium compounds;
oily or oil-soluble vitamins or vitamin derivatives that can be applied dermally: for example vitamin A (retinol in the form of the free acid or derivatives thereof), panthenol, pantothenic acid, folic acid, and combinations thereof, vitamin E (tocopherol), F; essential fatty acids; or niacinamide (nicotinic acid amide);
vitamin-based placenta extracts: active ingredient compositions comprising especially vitamins A, C, E, B₂₁, B₁₂, folic acid and biotin, amino acids and enzymes as well as compounds of the trace elements magnesium, silicon, phosphorus, calcium, manganese or copper;
skin repair complexes: obtainable from inactivated and disintegrated cultures of bacteria of the bifidus group;
plants and plant extracts: for example arnica, aloe, beard lichen, ivy, stinging nettle, ginseng, henna, camomile, marigold, rosemary, sage, horsetail or thyme;
animal extracts: for example royal jelly, propolis, proteins or thymus extracts;
cosmetic oils that can be applied dermally: neutral oils of the Miglyol 812 type, apricot kernel oil, avocado oil, babassu oil, cottonseed oil, borage oil, thistle oil, groundnut oil, gamma-oryzanol, rosehip-seed oil, hemp oil, hazelnut oil, blackcurrant-seed oil, jojoba oil, cherry-stone oil, salmon oil, linseed oil, cornseed oil, macadamia nut oil, almond oil, evening primrose oil, mink oil, olive oil, pecan nut oil, peach kernel oil, pistachio nut oil, rape oil, rice-seed oil, castor oil, safflower oil, sesame oil, soybean oil, sunflower oil, tea tree oil, rapeseed oil or wheatgerm oil.

The preparations in stick form are preferably anhydrous but may in certain cases comprise a certain amount of water which, however, in general does not exceed 40% by weight, based on the total weight of the cosmetic preparation.

If the cosmetic preparations and formulations according to the invention are in the form of semi-solid products, that is to say in the form of ointments or creams, they may likewise be anhydrous or aqueous. Such preparations and formulations are, for example, mascaras, eyeliners, foundations, make-up for the cheeks, eyeshadows, or compositions for treating rings under the eyes.

If, on the other hand, such ointments or creams are aqueous, they are especially emulsions of the water-in-oil type or of the oil-in-water type that comprise, apart from the pigment, from 1 to 98.8% by weight of the fatty phase, from 1 to 98.8% by weight of the aqueous phase and from 0.2 to 30% by weight of an emulsifier.

Such ointments and creams may also comprise further conventional cosmetic additives, for example perfumes, antioxidants, preservatives, gel-forming agents, UV filters, pearlescent agents, undyed polymers as well as inorganic or organic fillers.

If the preparations are in the form of a powder, they consist essentially of a mineral or inorganic or organic filler, for example talcum, kaolin, starch, polyethylene powder or polyamide powder, as well as adjuvants such as binders, etc.

Such preparations may likewise comprise various adjuvants conventionally employed in cosmetics, such as perfumes, antioxidants, preservatives, etc.

If the cosmetic preparations and formulations according to the invention are nail varnishes, they consist essentially of nitrocellulose and a natural or synthetic polymer in the form of a solution in a solvent system, it being possible for the solution to comprise other adjuvants, for example pearlescent agents.

In that embodiment, the inventive colorant mixture is present in an amount of approximately from 0.1 to 5% by weight.

The cosmetic preparations and formulations according to the invention are prepared in the conventional manner, for example by mixing or stirring the components together, optionally with heating, so that the mixtures melt.

In one embodiment the present invention also includes a method of coloring a cosmetic or personal care preparation, which comprises incorporating therein a tinctorially effective amount of a composition comprising at least one fluorescent colorant other than white and at least one non-fluorescent colorant.

In a preferred embodiment of the method the fluorescent colorant is selected from the group consisting of condensation products of p-N,N′-di(methyl)aminobenzaldehyde and barbituric acid, the benzoxanthene of the formula 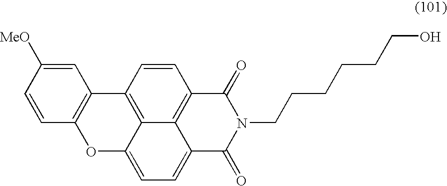
and mixtures thereof.

The present invention also includes a method of coloring human skin, which comprises applying to said skin a cosmetic or personal care preparation which comprises a tinctorially effective amount of at least one fluorescent colorant other than white and at least one non-fluorescent colorant.

In a preferred embodiment of this method the fluorescent colorant is selected from the group consisting of condensation products of p-N,N′-di(methyl)aminobenzaldehyde and barbituric acid, the benzoxanthene of the formula 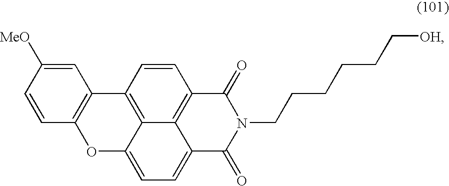
and mixtures thereof.

The following examples describe certain embodiments of this invention, but the invention is not limited thereto. It should be understood that numerous changes to the disclosed embodiments can be made in accordance with the disclosure herein without departing from the spirit or scope of the invention. These examples are therefore not meant to limit the scope of the invention. Rather, the scope of the invention is to be determined only by the appended claims and their equivalents. In these examples all parts given are by weight unless otherwise indicated.

### EXAMPLE 1

Lipstick with Fluorescent Organic Pigment

| Phase | INCI Name | Trade Name | Supplier | % |
|---|---|---|---|---|
| A | Castor Oil | Lipovol CO | Lipo | 45.10 |
| A | Octyldodecyl Stearoyl Stearate | Dermol 20-SS | Alzo | 12.90 |
| A | Carprylic/Capric Triglycerides | Pelemol CCT | Phoenix | 6.60 |
| A | Octyl Isononoate | Pelemol 89 | Phoenix | 2.70 |
| A | Mineral Oil | Mineral Oil | Univ. Preserve a | 1.30 |
| | | | chem | |
| A | Euphoria Cerifera | Candelilla Wax SP 75 | S & P | 6.50 |
| A | White Beeswax | White Beeswax | S & P | 3.90 |
| A | Carnauba Wax | Carnauba Wax SP63 | S & P | 0.70 |
| A | Ozokerite | Ozokerite | S & P | 2.90 |
| A | Methyl Glucose Sesquistearate | Glucate SS | Amerchol | 1.90 |
| A | Dodecyl Laurate, Lauryl Laurate | Purester-24 | S & P | 2.80 |
| B | Hydrogenated Polydecene | Silkflo 366NF | Lipo | 0.70 |
| B | Mica | 280 Mica Cosmetic, BC | Whittacker | 9.00 |
| B | Compound A* | Fluorescent organic | Ciba Specialty | 3.00 |
| | | colorant | Chemicals | |
| | | | Total | 100.00 |

*benzoxanthene of formula (101).

Procedure:

All the ingredients (Phase A) are melted together in a beaker at 80-85° C. using a propeller mixer until a uniform waxy base is formed. In a separate beaker, the ingredients of Phase B are combined and thoroughly mixed. The temperature of Phase A is lowered to about 72-75° C. and then Phase B is added with thorough mixing. The lipstick is poured into molds at 72-75° C. and then allowed to cool to room temperature.

### EXAMPLE 2

O/W Facial Foundation with Fluorescent Pigments

| Phase | INCI Name | Trade Name | Supplier | % |
|---|---|---|---|---|
| A | DI Water | DI Water | N/A | 68.72 |
| | TEA 99% | TEA 99% | | 0.20 |
| | PEG-12 Dimethicone | DC 193 | Dow | 0.10 |
| B | Butylene Glycol | Jeechem BUGL | Jeen | 4.00 |
| | Magnesium Aluminum | Veegum Granules OD-252 | R. T. Vanderbilt | 0.50 |
| | Silicate | | | |
| C | Butylene Glycol | Butylene Glycol | Jeen | 2.00 |
| | Cellulose | Sodium | Hercules | 0.10 |
| | | Carboxymethylcellulose | | |
| D | Methylparaben | Nipagin M | Clariant | 0.10 |
| E | Compound A* | Fluorescent organic colorant | Ciba Specialty Chemicals | 0.11 |
| | Condensate B** | barbituric acid condensate | Ciba Specialty Chemicals | 0.30 |
| | Encap. Red 7 Ca Lake | Encap. Red 7 Ca Lake | Ciba Specialty Chemicals | 0.38 |
| | Encap. Blue 1 Al Lake | Encap. Blue 1 Al Lake | Ciba Specialty Chemicals | 0.09 |
| F | Di-PPG-3 Myristyl | Promyristyl PM3 | Croda | 12.00 |
| | Ether Adipate | | | |
| | Dioctyl Maleate | Pelemol DOM | Phoenix Chemicals | 3.00 |
| | Steareth-10 | Lipocol S-10 | Lipo | 1.00 |
| | Steareth-2 | Lipocol S-2 | Lipo | 0.50 |
| | Cetyl Alcohol | Lipocol C | Lipo | 0.60 |
| | Crodafos CES | Crodafos CES | Croda | 4.00 |
| | Propyl Paraben | Nipasol M | Clariant | 0.10 |
| G | Talc | Altalc 400V | Whittacker | 2.00 |
| H | DMDM Hydantoin | Nipaguard DMDMH | Clariant | 0.20 |
| | | | Total | 100.00 |

*benzoxanthene of formula (101)
**condensation products of p-N,N'-di(methyl)aminobenzaldehyde and barbituric acid having the CAS numbers 1753-475 and 152734-34-4.

Procedure:

In a suitably sized beaker phase A is weighed out and mixed with a homogenizer for 5 minutes. Then heating the mixture is begun. Phase B is added to A and the mixture is homogenized for 30-40 minutes at 75-80° C. Phase C is added to the beaker and the mixture is homogenized for another 30 minutes at 75-80° C. Phase D is added and the mixture is mixed for 5 minutes. Phase E is added and the mixture is homogenized until the pigments are completely dispersed and no particles are seen floating throughout. In a separate beaker, phase F is melted at 75-80° C. and mixed well until a uniform liquid solution is obtained, which is then added to the heated mixture. The mixture is homogenized for 15 minutes at 75-80° C.; then cooling the mixture is begun. Phase G is added at 70° C. and the mixture is homogenized for 5-10 minutes, followed by paddle mixing. At 45° C. or below phase H is added. Slow mixing is stopped when the mixture reaches room temperature.

### EXAMPLE 3

### Comparative Example Showing Loss of Fluorescence with the Addition of Iron Oxide Colorants

O/W Facial Foundation with Fluorescent Pigments and Iron Oxides

| Phase | INCI Name | Trade Name | Supplier | % |
|---|---|---|---|---|
| A | DI Water | DI Water | N/A | 69.30 |
| | TEA 99% | TEA 99% | | 0.20 |
| | PEG-12 Dimethicone | DC 193 | Dow | 0.10 |
| B | Butylene Glycol | Jeechem BUGL | Jeen | 4.00 |
| | Magnesium Aluminum | Veegum Granules OD-252 | R. T. Vanderbilt | 0.50 |
| | Silicate | | | |
| C | Butylene Glycol | Butylene Glycol | Jeen | 2.00 |
| | Cellulose | Sodium | Hercules | 0.10 |
| | | Carboxymethylcellulose | | |
| D | Methylparaben | Nipagin M | Clariant | 0.10 |
| E | Compound A* | Fluorescent organic colorant | Ciba Specialty Chem. | 0.10 |
| | Condensate B** | barbituric acid condensate | Ciba Specialty Chem. | 0.10 |
| | Black Iron Oxide | C33-5198 | Sun Chemicals | 0.10 |
| F | Di-PPG-3 Myristyl | Promyristyl PM3 | Croda | 12.00 |
| | Ether Adipate | | | |
| | Dioctyl Maleate | Pelemol DOM | Phoenix Chemicals | 3.00 |
| | Steareth-10 | Lipocol S-10 | Lipo | 1.00 |
| | Steareth-2 | Lipocol S-2 | Lipo | 0.50 |
| | Cetyl Alcohol | Lipocol C | Lipo | 0.60 |
| | Crodafos CES | Crodafos CES | Croda | 4.00 |
| | Propyl Paraben | Nipasol M | Clariant | 0.10 |
| G | Talc | Altalc 400V | Whittacker | 2.00 |
| H | DMDM Hydantoin | Nipaguard DMDMH | Clariant | 0.20 |
| | | | Total | 100.00 |

*benzoxanthene of formula (101)
**condensation products of p-N,N'-di(methyl)aminobenzaldehyde and barbituric acid having the CAS numbers 1753-475 and 152734-34-4.

Procedure:

In a suitably sized beaker phase A is weighed out and mixed with a homogenizer for 5 minutes. Then heating the mixture is started. Phase B is added to A and the mixture is homogenized for 30-40 minutes at 75-80° C. Phase C is added to the beaker and the mixture is homogenized for another 30 min. at 75-80° C. Phase D is added and the mixture is mixed for 5 minutes. Phase E is added and the mixture is homogenized until the pigments are completely dispersed and no particles are seen floating throughout. In a separate beaker, phase F is melted at 75-80° C. and mixed well until a uniform liquid solution is obtained, which is then added to the heated mixture. The mixture is homogenized for 15 minutes at 75-80° C.; then cooling the mixture is begun. Phase G is added at 70° C. and the mixture is homogenized for 5-10 minutes. Then paddle mixing and cooling is begun. Phase H is added at 45° C. or below. Slow mixing is stopped when the mixture reaches room temperature.

The resulting facial foundation gives a colored preparation that is devoid of fluorescence due to the presence of iron oxide.

### EXAMPLE 4

Pressed Eyeshadow with Fluorescent Pigments

| Phase | INCI Name | Trade Name | Supplier | % |
|---|---|---|---|---|
| A | Mica | Sericite PHN | Presperse | 75.60 |
| A | Zinc Stearate | Zinc Stearate | Witco | 5.00 |
| A | Condensate B** | barbituric acid | Ciba Specialty | 6.00 |
| | | condensate | Chemicals | |
| A | Compound A* | Fluorescent organic | Ciba Specialty | 2.00 |
| | | colorant | Chemicals | |
| A | Methylparaben | Nipagin M | Clariant | 0.20 |
| A | Propylparaben | Nipasol M | Clariant | 0.10 |
| A | Calcium Aluminum Borosilicate | Luxil | Presperse | 5.00 |
| A | PEG-4 Diheptanoate | Liponate 2-DH | Lipo | 5.50 |
| | | | Total | 100.00 |

*benzoxanthene of formula (101)
**condensation products of p-N,N'-di(methyl)aminobenzaldehyde and barbituric acid having the CAS numbers 1753-475 and 152734-34-4.

Procedure:

The ingredients are combined and mixed well; then heated to 100° C. and pressed at 2000 psi.

### EXAMPLE 5

Preparation of Fluorescent Organic Colorant of Formula (101):

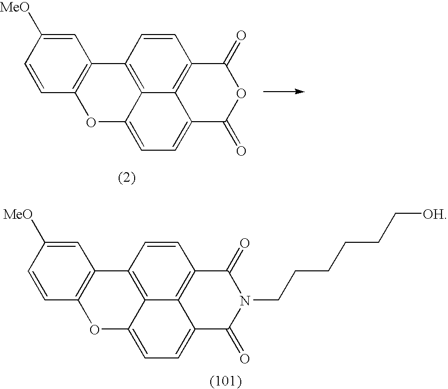

A mixture of the methoxybenzoxanthene derivative 2 [prepared according to literature methods disclosed in U.S. Pat. No. 3,741,971 to O. Fuchs et al.] (5 g, 0.0157 mol), 6-amino-1-hexanol (2.12 g, 0.0181 mol), toluene sulfonic acid monohydrate (0.21 g, 0.0011 mol) and N,N-dimethylformamide (200 ml) was stirred with heating to 135° C. under nitrogen for 18 hours, then cooled to 25° C. Methanol (20 ml) was then added, the mixture stirred for 30 minutes, then the yellow precipitate was filtered off, washed with methanol and then water and dried in vacuo at 40° C. Yield=4.88 g, 77%.

We claim:

1. A cosmetic or personal care composition, which comprises at least one fluorescent colorant other than white and at least one non-fluorescent colorant.

2. A composition according to claim 1, which provides a natural skin appearance.

3. A composition according to claim 1, wherein the fluorescent colorant is selected from the group consisting of pigments and dyes.

4. A composition according to claim 1, wherein the fluorescent colorant is selected from the group consisting of
a) perylene derivatives of the general formulae 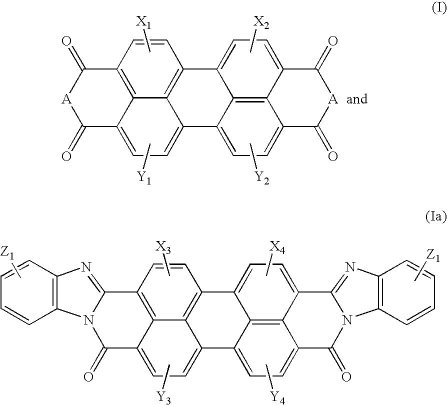
or its regioisomer,
wherein A=O, N—H, N-alkyl or N-aryl and X₁, X₂, X₃, X₄, Y₁, Y₂, Y₃, Y₄, Z₁, Z₂ are independently halogen, O-alkyl or O-aryl;
b) fluoresceins of the general formula 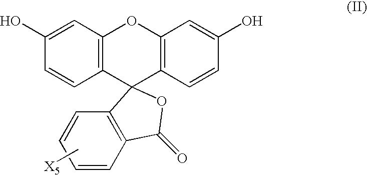
where X₅═Cl, Br or I;
c) rhodamines of the general formula 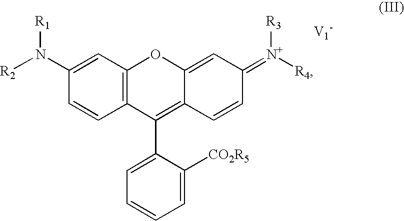
wherein the R₁, R₂, R₃, R₄, R₅ groups are independently H or C₁-C₁₂alkyl, and V₁═Cl, Br or I;
d) pyronines of the general formula 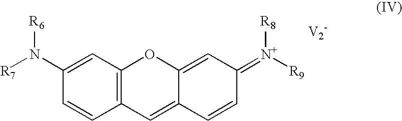
wherein R₆, R₇, R₈ and R₉ groups are independently H or C₁-C₁₂alkyl, and V₂═Cl, Br or I;
e) naphthalic anhydride derivatives of the general formula 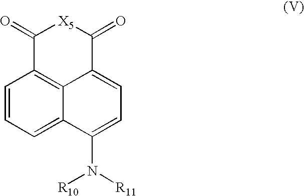
wherein X₅O, N—H, N-alkyl or N-aryl and R₁₀ and R₁₁ are independently H, alkyl or aryl;
f) benzoxanthenes and benzthioxanthenes of the general formulae 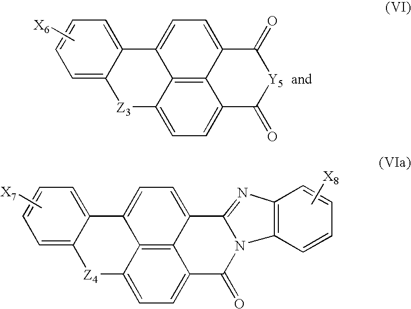
or its regioisomer,
wherein X₆, X₇ and X₈ are independently O-alkyl, O-aryl or halogen, Y₅═O, N—H, N-alkyl or N-aryl and Z₃ and Z are independently O or S;
g) benzanthrone derivatives of the general formula 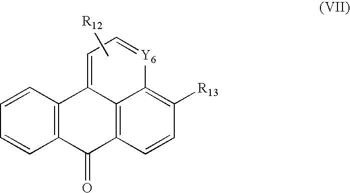
wherein Y₆═C or N and R₁₂ and R₁₃ are independently halogen, alkoxy, amino or alkylamino and when Y₆═C, Y₆ plus R₁₃ can form a 6-membered ring, optionally fused to an arylene group;
h) coumarins of the general formula 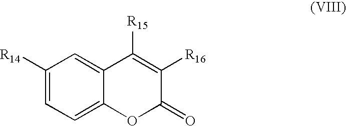
wherein R₁₄═H, alkoxy, amino or alkylamino, and R₁₅ and R₁₆ are independently H, alkyl or aryl;
i) isoindolo-[2,1-a]-benzimidazolone derivatives of the general formula 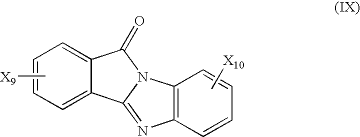
wherein X₉ and X₁₀ and are independently hydrogen or halogen;
j) diphenyl maleimides of the general formula 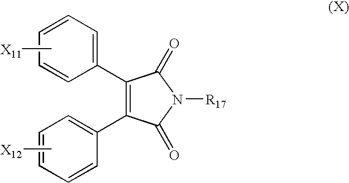
wherein R₁₇=optionally substituted alkyl or aryl and X₁₁, and X₁₂ are independently H, alkyl, aryl, O-alkyl, O-aryl or halogen;
k) azo pigments of the general formula 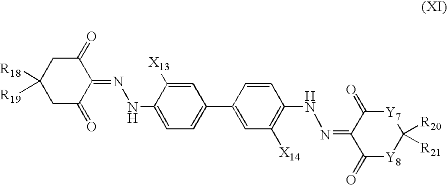
wherein X₁₃ and X₁₄ are independently halogen, alkoxy, Y₇ and Y₈ are independently CH₂ or 0 and R₁₈, R₁₉, R₂₀ and R₂₁ are independently optionally substituted alkyl or aryl;
l) diketopyrrolo[3,4-c]pyrroles of the general formula 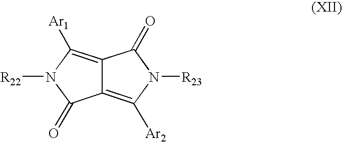
wherein Ar₁ and Ar₂ are aryl radicals and R₂₂ and R₂₃ are optionally substituted alkyl or aryl radicals;
m) condensation products of aromatic or heteroaromatic aldehydes with barbituric acid derivatives,
and mixtures thereof.

5. A composition according to claim 1, wherein the fluorescent colorant has a yellow and/or reddish hue.

6. A composition according to claim 4, wherein the fluorescent colorant is selected from the group consisting of condensation products of N,N′-di(C₁-C₈alkyl)aminobenzaldehyde and barbituric acid, perylene derivatives of the formula (I) or (Ia), diphenyl maleimides of the formula (X), benzoxanthenes and benzthioxanthenes of the formulae (VI) and (Via), N,N′-disubstituted DPPs of the formula (XII) and the isoindolo-[2,1-a]-benzimidazolone of the formula 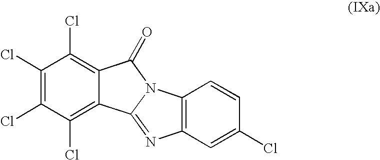
and mixtures thereof, wherein the substituents in the formulae are as defined in claim 4.

7. A composition according to claim 4, wherein the fluorescent colorant is selected from the group consisting of condensation products of p-N,N′-di(methyl)aminobenzaldehyde and barbituric acid, the benzoxanthene of the formula 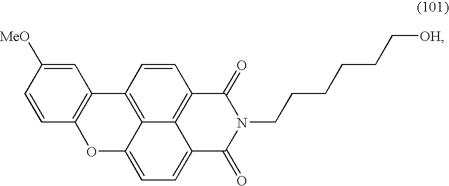
and mixtures thereof.

8. A composition according to claim 1, wherein the non-fluorescent colorant is a dye, pigment or lake, or a mixture thereof.

9. A cosmetic or personal care preparation or formulation, which comprises from 0.005 to 20% by weight, based on the total weight of the preparation of a mixture of at least one fluorescent colorant other than white and at least one non-fluorescent colorant and a cosmetically acceptable adjuvant.

10. A preparation according to claim 9, which comprises 0.005 to 2% by weight of the fluorescent colorant.

11. A preparation according to claim 9, which comprises 0.005 to 20% by weight of the non-fluorescent colorant.

12. A preparation according to claim 9, which is in the form of a lipstick, mascara preparation, make-up for the cheeks, eyeshadow, foundation, eyeliner, facial or body powder or nail polish.

13. A preparation according to claim 9, which comprises additional cosmetically acceptable active ingredients.

14. A preparation according to claim 9, wherein the cosmetically acceptable active ingredients are glycols, polyethylene glycols, polypropylene glycols, monoalkanolamides, undyed polymeric, inorganic or organic fillers, preservatives, UV filters and reflectors or other adjuvants and additives conventionally employed in cosmetics.

15. A method of coloring a cosmetic or personal care preparation, which comprises incorporating therein a tinctorially effective amount of a mixture of at least one fluorescent colorant other than white and at least one non-fluorescent colorant according to claim 1.

16. A method according to claim 15, wherein the fluorescent colorant is selected from the group consisting of condensation products of p-N,N′-di(methyl)aminobenzaldehyde and barbituric acid, the benzoxanthene of the formula 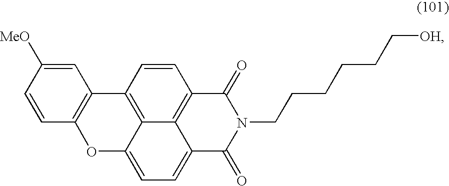
and mixtures thereof.

17. A method of coloring human skin, which comprises applying to said skin a cosmetic or personal care preparation according to claim 9.

18. A method according to claim 17, wherein the fluorescent colorant is selected from the group consisting of condensation products of p-N,N′-di(methyl)aminobenzaldehyde and barbituric acid, the benzoxanthene of the formula 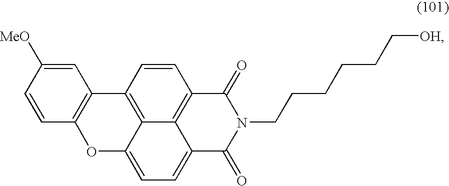
and mixtures thereof.

19. A method of preparing a colored cosmetic or personal care preparation according to claim 9, which comprises incorporating into said preparation a tinctorially effective amount of a mixture of at least one fluorescent colorant other than white and at least one non-fluorescent colorant.

20. The benzoxanthene of the formula [chemical structure]
